(19) United States
(12) Patent Application Publication
(10) Pub. No.: US 2001/0007020 A1
(43) Pub. Date: Jul. 5, 2001

(54) ANTIBODIES THAT BIND TO THE NIDOGEN-BINDING DOMAIN OF LAMININ, THEIR PRODUCTION AND USE

(75) Inventor: MARTIN GERL, NIEDERNHAUSEN (DE)

Correspondence Address: FINNEGAN HENDERSON FARABOW, GARRETT & DUNNER, 1300 I STREET NW, SUITE 700, WASHINGTON, DC, 200053315

(73) Assignee: HOECHST AKTIENGESELLSCHAFT

(21) Appl. No.: 09/341,643

(22) PCT Filed: Dec. 22, 1997
(86) PCT No.: PCT/EP97/07241

§ 371 Date: None

(30) Foreign Application Priority Data
Jan. 17, 1997 (DE) 19701607.3

(51) Int. Cl. C07K 16/00

(52) U.S. Cl. 530/388.1; 530/389.1

(57) ABSTRACT

The invention relates to monoclonal and polyclonal antibodies, and their parts, which bind specifically to the nidogen-binding domain of laminin, to processes for their preparation and to their use as pharmaceuticals, as diagnostic agents for detecting laminin isoforms and as model substances for developing and evaluating substances which affect the nidogen/laminin interaction. The antibodies according to the invention, or their parts, bind preferentially to the laminin γ1 III 4 domain, in particular in the highly conserved region of the a and c loops, and are able to inhibit the association of laminin and nidogen.

The invention relates to monoclonal and polyclonal antibodies, and parts thereof, which bind specifically to the nidogen-binding domain of laminin, to processes for their preparation and to their use as pharmaceuticals, as diagnostic agents for detecting laminin isoforms and as model substances for developing and evaluating substances which influence the nidogen/laminin interaction. The antibodies according to the invention, or parts thereof, preferably bind to the γ1 III 4 domain of laminin, in particular to essential nidogen-binding sites in the highly conserved region of loop a or of loops a and c, and are able to inhibit the association of laminin and nidogen.

The association of laminin (an 800 kDa glycoprotein) and nidogen (a 160 kDa glycoprotein) is regarded as being a crucial biomolecular mechanism in the synthesis and stabilization of basement membranes (Mayer, U. & Timpl, R. (1994) in: Extracellular Matrix Assembly and Structure (Ed.: P. D. Yurchenco et al.) pp. 389-416, Academic Press, Orlando, Fla.). Due to its ability to form ternary complexes with all the main constituents of the basement membrane, such as γ1-containing laminin isoforms (for nomenclature, see: Burgeson, R. E. et al. (1994) Matrix Biology 14:209-211), collagen IV, perlecan and fibulin, and their respective association structures, nidogen assumes the function of a linking member which interconnects, spatially organizes and stabilizes the mutually differing macrostructures (Fox, J. W. et al. (1991) EMBO J. 10:3137-3146; Aumailley, M. et al. (1993) Kidney Int. 43:7-12).

Experiments using polyclonal anti-laminin antibodies provided clear evidence that the laminin/nidogen interaction plays a central role in the synthesis of a functional basement membrane. These antibodies were obtained by immunizing rabbits with laminin P1 or the recombinantly produced laminin fragment γ1 III 3-5, and were concentrated by means of affinity chromatography on laminin P1 or laminin γ1 III 3-5 matrices. In inhibition tests, they completely inhibited the laminin/nidogen association. However, this inhibition is based on the antibodies, whose binding regions are only located in the vicinity of the nidogen-binding sequences of the laminin, sterically blockading the access of the nidogen to the laminin (Mayer, U. et al. (1993) EMBO J. 12:1879-1885). In embryonic organ cultures, these antibodies were able to inhibit both the genesis of renal tubules and the formation of pulmonary alveoli. Both of these processes are ontogenesis programs which depend on the unimpeded neosynthesis of a basement membrane (Ekblom, P. et al. (1994) Development 120:2003-2014; Ekblom, P. (1993) in: Molecular and Cellular Aspects of Basement Membranes (Ed.: Rohrbach, D. H. & Timpl, R.) pp. 359-383; Academic Press, San Diego, Calif.).

The nidogen-binding domain of laminin has been unambiguously identified and characterized with regard to its location and sequence and with regard to its spatial structure (X-ray crystal structure and NMR structure) (Mayer, U. et al. (1993) EMBO J. 12:1879-1885; Baumgartner, R. et al. (1996) J. Mol. Biol. 257:658-668; Stetefeld, J. et al. (1996) J. Mol. Biol. 257:644-657). It is located in an “LE module” (laminin-type, epidermal growth factor-like) in the γ1 III 4 domain of the short arm of the γ1 chain of the laminin. “LE modules” are structural motifs which are composed of 50-60 amino acids and which exhibit a complex folding pattern which is analogous to that of epidermal growth factor and which possesses 4 disulfide bridges (Bairoch, A. (1995) Nomenclature of extracellular domains. The SWISS-PROT Protein sequence data bank. Release 310; Engel, J. (1989) FEBS Letters 251:1-7).

It has been demonstrated that nidogen binds with high affinity to the complementary laminin domain in the case of mouse EHS tumor laminin P1, in the case of human placental laminin 2 and laminin 4, and in the case of Drosophila laminin. The reason for this species-overlapping binding specificity is the extraordinarily high degree of amino acid sequence identity which exists in the laminin γ1 III 4 domain in the species investigated. It amounts to 97% between humans and mice and an astonishing 61% between humans and Drosophila when the whole module is taken into account. If the comparison is restricted to the region of the a to c loops, which contain the essential nidogen-binding sites, these values then increase to 100% and 75%, respectively (Pikkarinen, T. et al. (1987) J. Biol. Chem. 263:6751-6758; Chi, H.-C. & Hui, C.-F. (1989) J. Biol. Chem. 264:1543-1550).

In addition to demonstrating that the binding of the nidogen depended on an intact three-dimensional structure, it was also possible to identify well-defined sequence regions which are located in the S-S-stabilized a and c loops of the laminin γ1 III 4 domain. Five essential amino acids were identified: four are located within a segment of 7 amino acids in the a loop and a tyrosine side chain in the c loop (Pöschl, E. et al. (1994) EMBO J. 13:3741-3747; Mayer, U. et al. (1993) EMBO J. 12:1879-1885; Pöschl, E. et al. (1996) EMBO J. 15:5154-5159).

Synthetic peptides, which can be derived from the corresponding regions of the laminin γ1 III 4 domain, are able to completely inhibit laminin/nidogen binding in special binding assays (U.S. Pat. No. 5,493,008). However, such synthetic peptides exhibit an activity, in inhibition assays, which is about 400-10,000 times lower than that of intact laminin P1 or laminin γ1 III 3-5 (Pöschl, E. et al. (1994) EMBO J. 13:3741-3747; U.S. Pat. No. 5,493,008). The laminin/nidogen interaction is affected by a powerful conformational component (Mayer, U. et al. (1993) EMBO J. 12:1879-1885). The weaker inhibitory effect of the synthetic peptides can be explained on the basis that, in aqueous solution, peptides are able to adopt a myriad of different conformations and, as a result, only a certain percentage of the peptides is present in the biologically active conformation.

The use of these peptides as medicaments is therefore subject to substantial limitations due to their confirmational flexibility and also on account of their instability toward proteases and their poor bioavailability and pharmacodynamics (Milner-White, E. J. (1989) Trends Pharmacol. Sci. 10:70-74; Hruby, V. J. (1994) in: Peptides, Proc. Thirteenth American Peptide Symposium; (Ed.: Hodges, R. S. & Smith, J. A.) pp. 3-17; ESCOM: Leiden, Netherlands).

Antibodies which bind specifically to the nidogen-binding domain of the laminin, and which are able to competitively inhibit, at low concentration, the association between laminin and nidogen, are more suitable, due to their higher affinity and avidity, their high degree of stability and their satisfactory pharmacokinetics, for use as therapeutic agents for treating diseases. Furthermore, they can be used as diagnostic agents or as aids in biological and pharmacological models for developing and evaluating substances which affect the laminin/nidogen interaction.

While some of the anti-laminin P1 or anti-laminin γ1 III 3-5 antibodies which have previously been produced are able to inhibit nidogen/laminin binding, they do not recognize the nidogen-binding sites of the laminin γ1 chain directly; instead, the nidogen/laminin binding is inhibited as the result of a steric interaction (Mayer, U. et al. (1993) EMBO J. 12:1879-1885). The nidogen-binding domain of the laminin γ1 chain is extraordinarily strongly conserved in a species-overlapping manner. In addition to this, laminin is an extracellular protein which is in constant contact with the immune system, both as an integrated constituent of basement membranes and in the form of a circulating serum component (EP 0 696 597 A2). Because the immune system is able to distinguish “self” from “non-self”, it must be concluded that each immunized species recognizes the highly conserved immunization antigen as being a constituent of its own body and for this reason does not develop any antibodies against this constituent. The production of a specific antibody titer was not therefore to be expected. This generally recognized doctrine has been confirmed by the fact that it has so far not been possible to produce any antibodies against the laminin nidogen-binding domain by immunizing rabbits with laminin P1 and laminin γ1 III 3-5 (Mayer, U. et al. (1993) EMBO J. 12:1879-1885). However, the above-described polyclonal antibodies, which bind to imprecisely defined epitopes lying outside the nidogen-binding domain of the laminin, are of only very limited suitability, or are completely unsuitable, for use as therapeutic agents, as diagnostic agents or as model substances for developing and evaluating substances which affect the nidogen/laminin interaction: since steric inhibition depends on the spatial extent of the inhibitor, it is scarcely possible to use parts of these antibodies as therapeutic agents, as would be preferred for pharmacological reasons. Furthermore, possible cross reactions with analytes which are not to be detected restrict the use of these antibodies in diagnostic tests.

The object of the present invention is to produce antibodies which bind specifically to the nidogen-binding domain of laminin, that is, which directly recognize the nidogen-binding domain of the laminin γ1 chain, and which are suitable for use as pharmaceuticals, as diagnostic agents for detecting laminin isoforms and as model substances for developing and evaluating substances which affect the nidogen/laminin interaction.

The object is achieved, in accordance with the invention, by the antibodies which are described below and by the processes for preparing and using them.

The antibodies according to the invention, or parts thereof, characteristically bind to the nidogen-binding domain of laminin, i.e. the laminin γ1 III 4 domain, preferably to the highly conserved region of the a loop or of the a and c loops of the laminin γ1 III 4 domain. Particularly preferably, the antibodies according to the invention bind, in a conformation-dependent manner with regard to the epitope (i.e. recognizing the nidogen-binding site of the laminin in its native conformation; cf. Example 6), directly or in an overlapping manner, to the highly conserved region of the a loop or of the a and c loops. In particular, the invention includes antibodies, or parts thereof, which bind at least to a peptide as depicted in Table 1. The present invention provides both polyclonal and monoclonal antibodies. The antibodies according to the present invention are preferably chimeric, humanized, bispecific or oligospecific antibodies. Particularly preferably, the laminin/nidogen binding is inhibited competitively or partially competitively by the antibodies according to the present invention (cf. Example 7).

Table 1: Amino acid sequences of the peptides used for immunization

(1): DNIDPNAVGNL

(2) DNIDPNAVGNLKCIYNTAGFYCDR (S-S-bridged-form)

|______|

The antibodies according to the invention can be obtained by immunizing immunocompetent vertebrates, such as rabbits, mice, sheep, goats, guinea pigs, rats and hens, with laminin, laminin P1, laminin γ1 III-3-5 or laminin γ1 III 4, and also, in particular, with peptides which comprise essential nidogen-binding sites but not the complete amino acid sequence of the γ1 III 4 domain of laminin, very particularly preferably one or both of the peptides depicted in Table 1, as the immunizing antigen.

When the immunization is carried out with laminin or laminin P1, the antibody is identified using laminin γ1 III-3-5 and/or laminin γ1 III-4 and is finally tested for its ability to inhibit the laminin/nidogen binding competitively or partially competitively.

When the immunization is carried out with laminin γ1 III-3-5, the antibody is identified using laminin and/or laminin P1 and is finally tested for its ability to inhibit the laminin/nidogen binding competitively or partially competitively.

Particular preference is given to using laminin γ1 III-4 or one or more peptides depicted in Table 1 as the immunizing antigen(s). The antibodies which can be obtained by immunizing with these immunizing antigens are preferably identified using laminin and/or laminin P1. Advantageously, the identified antibodies are tested for their ability to inhibit the laminin binding site competitively or partially competitively.

Besides polyclonal antibodies, monoclonal antibodies (Mabs) can also be obtained, with, in the latter case, Mab-producing hybridoma cells being produced initially. The antibodies can also be obtained in purified form, with affinity chromatography, preferably on laminin and/or laminin P1 as the affinity matrix, being, for example, used to purify the antibodies according to the invention from antibody-containing material, such as the antiserum of the immunized animal, a hybridoma cell culture supernatant, ascites or cells.

The antibodies according to the invention, or parts thereof, are able to inhibit the laminin/nidogen interaction and also comprise, as a collective term, the corresponding chimeric, humanized, bispecific or oligospecific antibodies, and also antibody analogs, which are described in more detail elsewhere.

The invention also comprises animal, plant and prokaryotic cells, and also cell lines, which produce the antibodies and antibody parts according to the invention, preferably the hybridon DSMACC 2327, which was deposited in the Deutsche Sammiung von Mikroorganismen und Zelikulturen GmbH (German Collection of Microorganisms and Cell Cultures) (DSMZ, Marscheroder Weg 1b, D-38124 Braunschweig, Germany) on Oct. 27, 1997 in accordance with the provisions of the Budapest treaty. The present invention also relates to the monoclonal antibody which is produced by the hybridoma which is deposited under deposition number DSMACC 2327.

The invention furthermore comprises a process for preparing the above-described antibodies, with immunocompetent vertebrates, such as rabbits, mice, sheep, goats, guinea pigs, rats and hens, [lacuna] with laminin, laminin P1, laminin γ1 III 3-5 and laminin γ1 III 4, particularly preferably [lacuna] peptides which do not contain the complete amino acid sequence of the laminin γ1 III 4 domain, very particularly preferably one or both of the peptides depicted in Table 1. The term “peptides” is to be understood as meaning oligopeptides, polypeptides and also proteins and protein fragments. When used as immunizing antigens, the peptides are preferably employed coupled to carriers such as proteins, e.g. ovalbumin, albumin or hemocyanin, or polymers, e.g. polyethylene glycol, polyacrylamide or poly-d-glutamine-d-lysine.

When the immunization is carried out with laminin or laminin P1, the antibody is identified using laminin γ1 III-3-5 and/or laminin γ1 III-4 and tested for its ability to inhibit the laminin/nidogen binding competitively or partially competitively.

When the immunization is carried out with laminin γ1 III-3-5, the antibody is identified using laminin and/or laminin P1 and tested for its ability to inhibit the laminin/nidogen binding competitively or partially competitively.

Laminin γ1 III-4 or, as desired, one or both of the peptides depicted in Table 1, which is/are preferably coupled to a carrier, is/are preferably employed in the process according to the invention.

The antibody which is produced by immunizing with laminin γ1 III-4 or with one or both the peptides depicted in Table 1 is preferably identified using laminin and/or laminin P1 and advantageously tested for its ability to inhibit the laminin/nidogen binding competitively or partially competitively.

The process also optionally comprises generating MAb-producing hybridoma cells. It has proved to be advantageous to purify the antibodies according to the invention, or parts thereof, from antibody-containing material such as the antiserum of the immunized animal, a hybridoma cell culture supernatant, ascites or cells, for example with the aid of affinity chromatography, with a laminin and/or laminin P1 affinity matrix preferably being used.

The antibodies according to the invention, or parts thereof, can be used in many different ways, for example as pharmaceuticals, as diagnostic agents, as aids in biological and pharmacological models for developing and evaluating substances which affect the laminin/nidogen interaction, for example as model substances for evaluating the spatial structure of the contact zone which is complementary to the nidogen-binding site of laminin, and the potential binding valencies of this contact zone, and for investigating the biosynthesis of basement membranes and the influence of basement membranes in different physiological processes such as organ development, angiogenesis or embryogenesis. The invention also includes pharmaceuticals and diagnostic agents which comprise one or more of the antibodies or antibody parts according to the invention.

The invention also comprises the use of one or more antibodies or antibody parts according to the invention

for preparing a pharmaceutical for treating diseases in which there is an increased or undesirable synthesis of basement membranes, in particular fibroses, especially alcoholic hepatic fibrosis and pulmonary fibrosis, and also all forms of diabetic late complications which are accompanied by thickenings of the basement membrane—especially in the kidney, the eye and the vascular system—, and also arteriosclerosis and all diseases in which angiogenesis contributes to aggravation of the clinical picture, e.g. cancer diseases, diabetic retinopathy and diseases having a strong inflammatory component, such as rheumatoid arthritis, osteoarthritis, vasculitis, hemangiomas and psoriasis;

for preparing a diagnostic agent for detecting γ1-containing laminin isoforms in biological samples, e.g. in body fluids such as blood, serum, plasma, urine, saliva or cerebrospinal fluid, and also in tissues. The term diagnostic agent includes, for example, the different embodiments of heterogeneous and homogeneous immunoassays, test systems in immunohistochemistry and reagents for in-vivo detection methods such as immunoscintigraphy. The preparations comprising the antibodies or antibody parts according to the invention can also be combined in the form of diagnostic kits either alone or together with further auxiliary reagents, such as buffers, washing solutions, measurement signal-emitting solutions, and/or other aids, such as cuvettes.

In that which follows, the invention is described in more detail and clarified with the aid of various examples:

Surprisingly, it was possible to produce antibodies against the highly conserved amino acid sequence of the nidogen-binding domain of laminin using the peptides which are listed in Table 1 and which are unable to form any folding pattern as seen in LE modules. For this, the peptides depicted is Table 1 were coupled to ovalbumin using carbodiimide and these conjugates were then employed to immunize rabbits. The development of a specific antibody titer against laminin P1 and laminin γ1 III 3-5 was analyzed with the aid of an enzyme immunoassay.

The polyclonal antibodies of desired specificity were then concentrated by subjecting them to affinity chromatography through a matrix to which human placental laminin P1 was bound and then to molecular sieve chromatography. The methods for purifying and characterizing human placental laminin, and its use for immunizing mice and for isolating anti-laminin P1 antibody-synthesizing hybridomas are described in EP 0 696 597 A2.

The antibodies which are concentrated after subjecting the antiserum to laminin P1 affinity chromatography display binding specificity toward human placental laminin P1, mouse laminin P1 (EHS tumor) and rat laminin (yolk sac). In addition to recognizing the specific sequence, the antibodies also recognize the biologically active conformation of the nidogen-binding domain of laminin. They are able to inhibit laminin/nidogen binding completely.

However, the antibodies according to the invention can also be obtained by preferably immunizing other immunocompetent vertebrates, such as mice, sheep, goats, guinea pigs, rats and chickens, with laminin γ1 III 4 and also, in particular, with peptides which contain important nidogen-binding sites but do not contain the complete amino acid sequence of the laminin γ1 III 4 domain, very particularly preferably the peptides depicted in Table 1. Polyclonal antibodies according to the invention can be purified from the antiserum of the immunized animals. In order to produce corresponding monoclonal antibodies, the immune cells of immunized animals, such as mice, are fused with myeloma cells in order to produce Mab-producing hybridoma cells and suitable clones are then isolated, using well known methods (see, for example, Harlow, E. & Lane, D. (1988) Antibodies: A Laboratory Manual, Cold Spring Harbor Laboratory, Cold Spring Harbor). The desired Mab-producing clones are selected using specific screening methods. Here, enzyme immunoassays or radioimmunoassays, and also Western blots, are preferably used to examine the specificity of the antibodies which are released into the culture supernatant for binding, for example, to the immunizing antigen, to the immunizing antigen carrier, and to native and recombinant laminin and/or its fragments. A further possible selection criterion is the ability of the antibodies to prevent nidogen/laminin binding. This ability can be evaluated, for example, using the inhibition assays which are described in detail in the examples. Hybridomas which produce Mabs which bind specifically to the nidogen-binding domain of laminin are cloned. They are then available for the long-term production of the Mabs. Depending on the desired purpose, it may be advantageous only to use parts of the antibodies, such as F(ab)₂, Fab′ or Fab fragments. These can be produced, for example, using enzyme cleavages methods which are known to the skilled person (see, for example, Harlow, E. & Lane, D. (1988) Antibodies: A Laboratory Manual, Cold Spring Harbor Laboratory, Cold Spring Harbor).

The antigen-binding sites of an antibody are located in the so-called variable domains, which are encoded by the corresponding V genes. The known genetic manipulation methods (see, for example, Sambrook, J. et al. (1989) Molecular Cloning: A Laboratory Manual, Cold Spring Harbor Laboratory, Cold Spring Harbor, 2nd. edition; McCafferty, J. et al. (1990) Nature 348:552-554) can also be used to determine the corresponding nucleic acid sequence of an antibody which binds to the nidogen-binding domain of laminin and thereby the corresponding amino acid sequence as well, provided this sequence is not already known as a consequence of carrying out amino acid sequencing. Hybridoma cells or the antibody-producing immune cells of immunized mammals are employed as the starting material for the analyses.

With the nucleic acid and amino acid sequences being known, customary genetic manipulation and molecular biological methods (see also Johnson, K. S. & Chiswell, D. J. (1993) Current Opinion in Structural Biology 3:564-571) can then be used to prepare humanized or chimeric, bispecific or oligospecific antibodies and also antibody analogs, such as peptides (“minimal recognition units”) which are derived from the complementarity determining region, single-chain fragments and functional fusion products, such as, in particular, recombinantly prepared antibody/enzyme or antibody/complement constructs, which bind specifically to the nidogen-binding domain of laminin. These molecules which are derived from the antibody or antibody gene according to the invention are included in the overall term “antibody or a part thereof” which is used in this present document. These molecules can be used, for example, to achieve a decrease in immunogenicity and/or an increased efficacy when they are administered as pharmaceuticals, and/or advantages ensue when they are used as diagnostic agents or as aids for developing and evaluating substances which affect the laminin/nidogen interaction. The antibodies or parts thereof can be prepared in plant (e.g. yeast), animal and prokaryotic cells.

The antibodies according to the invention, and parts thereof, can be modified, for example by labeling them with radioactive isotopes or paramagnetic compounds when they are to be used for in-vivo diagnosis, or by bonding pharmacologically active substances to them in order to produce an even more effective pharmaceutical.

The examples which are cited below serve to clarify individual aspects of the invention by way of example.

### EXAMPLES

SDS gel electrophoresis, Western blotting and BCA protein determination were carried out in accordance with standard protocols or the manufacturers' instructions. When not expressly indicated, the chemicals employed were obtained from Merck (Darmstadt), Sigma (Munich) or Riedel de Haen (Seelze).

### Example 1: Synthesis of peptides

148 mg (0.1 mmol) of an FMOC-amide anchor-PAM resin were used for the solid phase synthesis—ABI 433 peptide synthesizer—of the peptide. After the synthesis had been completed, the weight of the resin was observed to have increased to 513 mg. The resin was treated, at room temperature for 2 hours, with a solution composed of 10 ml of trifluoroacetic acid and 365 μl of triethylsilane. After the resin had been filtered off, the solution was concentrated by rotary evaporation in vacuo and the residue was taken up in 50 ml of 10% acetic acid (AcOH), and this solution was then freeze-dried. 145 mg (54% yield) of crude peptide were obtained. The crude peptide was then dissolved in 3 ml of 80% AcOH, and this solution was added dropwise to a solution (0.006 mmol of iodine and 0.006 mmol of sodium acetate in 55 ml of 80% AcOH) which was being stirred rapidly. After 5 min, the reaction was terminated by adding an 0.1 N solution of ascorbic acid. The solution was concentrated down to a volume of 2 ml and loaded onto a Sephadex® G25 column, which was developed with 0.1 M AcOH. Reversed phase HPLC chromatography was then used to obtain the isolated peptide in highly purified form.

Yield: 36 mg of DNIDPNAVGNLKCIYNTAGFYCDR-NH₂ (13.5% of theory.)

The structure was confirmed by mass spectroscopy (molar mass: 2673 Da) and amino acid analysis.

The peptide DNIDPNAVGNL-NH₂ was prepared in an analogous manner.

Yield: 268 mg (67% of theory); molar mass: 1140 Da

### Example 2: Coupling the peptides to ovalbumin

30 mg of ovalbumin (Sigma A 2512) were dissolved in 1 ml of Na phosphate buffer, pH 7.4, and 200 μl of an aqueous solution of 7 mg of N-hydroxysulfosuccinimide Na salt (Fluka 56485) and 300 μl of an aqueous solution of 100 mg of 1-ethyl-1-3-(3-diaminaminopropyl)carbodiimide, HCl (Sigma E 6383) were then added to this ovalbumin solution. After 5 minutes, the peptide solution (30 mg of the appropriate peptide in 1 ml of 10 mM Na phosphate buffer, pH 7.4) was added. The coupling reaction proceeded for 16 hours at room temperature in the dark. At the end of the reaction period, the solution was centrifuged in order to remove any turbidity which might have arisen. Unreacted chemicals and salts were then removed by chromatography through an NAP 25 column (Pharmacia). This transferred the ovalbumin/peptide conjugate into PBS+0.04% Tween 20. The yield was 50-55 mg of conjugate.

Conjugate 1: ovalbumin-DNIDPNAVGNL

Conjugate 2: ovalbumin-DNIDPNAVGNLKCIYNTAGFYCDR (S-S-bridged form)

|______|

### Example 3: Immunization of rabbits

Mixed-race rabbits having a body weight of approx. 3 kg were immunized with the ovalbumin/peptide conjugates. For this, 1 mg of each of the appropriate conjugates was dissolved in 0.5 ml of phosphate-buffered saline (PHS) and this solution was then mixed with the same volume of complete Freund's adjuvant and the whole was carefully emulsified. 1 ml of the resulting emulsion was injected intradermally into a rabbit; in this operation, one fifth of the volume was in each case administered to one of five different sites in the vicinity of the regional lymph nodes. Booster injections (for these, the antigen was emulsified with incomplete Freund's adjuvant) with half-concentrated antigen emulsion were given after 21 days and 53 days.

### Example 4: Titer development in four immunized rabbits

The development of specific antibody titer was investigated in detail in four animals by means of an enzyme immunoassay using laminin P1-coated or laminin γ1 III 3-5-coated plastic receptacles. The corresponding animal sera are specified by the designations R2, R3, R4 and R905. The sera R3 and R4 were obtained by immunizing with conjugate 1, while the sera R2 and R905 were obtained by immunizing with conjugate 2. An immune reaction was very rapidly stimulated in all the rabbits, with this immune reaction then remaining constant after 21 days (1st booster) or declining slowly but continuously. The immune response was not observed to be restimulated by a second booster injection. The antibodies which were formed reacted both with laminin P1 and with the laminin γ1 III 3-5 domain. Nevertheless, the binding to laminin P1 was somewhat less pronounced than that to laminin γ1 III 3-5 and appeared to be subject to greater fluctuations in the process of the immune reaction. This may indicate the presence in the polyclonal serum of several antibody populations which bind the nidogen-binding motifs, or their conformations in laminin P1 and laminin γ1 III 3-5, with differing affinities.

### Example 5: Affinity purification of the antibodies

For the purpose of further characterization, the specific antibodies were separated from the remaining immunoglobulins in the animal serum, from other serum constituents and from the antibodies which were directed against the ovalbumin. To do this, an affinity chromatography was carried out on a laminin P1 affinity matrix. Fractogel® EMD azlactone 650(S) (Merck, Darmstadt) was used as the support (gel matrix). Prior to the coupling, 0.3 g of material was incubated for 15 minutes in 6 ml of PBS, 1 M Na₂SO₃, pH 7.4, and the liquid supernatant was then poured off. During the incubation, the material swelled to a volume of 1 ml and it was possible to use the matrix directly for the covalent coupling of the desired ligand.

3.4 mg of human laminin P1 were dissolved in 2 ml of 0.2 M ammonium bicarbonate, pH 8.0, and this solution was incubated at 4° C. overnight (>16 h) with 1 ml of activated (see above) support material. The gel material was then washed with 0.2 M ammonium bicarbonate, pH 8.0. The remaining active groups on the support material were blocked by a 24-hour incubation at 4°C. in 5 ml of 0.2 M glycine, pH 8.0. The gel matrix was prepared finally for the affinity binding by means of three washing cycles comprising alternating incubations in PBS, 1 M Na₂SO₃, pH 7.4, 0.1 M Na acetate, pH 4.0, and 0.2 M glycine, pH 8.0. A Pharmacia HR 5/5 column was filled with the matrix, which was then equilibrated with PBS/0.04% Tween 20.

In order to purify laminin P1-binding antibodies from the animal sera, the relevant serum was diluted 1:2 with PBS/0.04% Tween 20 and passed through the column at a flow rate of 2 ml/min. The column was then subsequently washed with buffer until the base line had once again been reached in the flow-through monitor UV signal (220 nm). The bound antibodies were finally eluted by changing the running buffer to 0.1 M glycine/HCl, pH 2.7.

The column flow-throughs and column eluates were analyzed by means of an enzyme immunoassay using immobilized laminin γ1 III 3-5.

The above-described affinity purification was successfully used to purify antibodies from the four animal sera. The average yield was 0.3 mg per 50 ml of serum (the yield was only 0.1 mg in the case of serum R2). However, the majority of the antibodies in all the sera (>80%) did not bind to laminin P1, presumably either due to the binding kinetics being too slow or due to an exclusive affinity for the sequence-of the peptide which was used for the immunization.

Affinity chromatography on laminin P1 columns consequently leads to selective enrichment of the rapidly and stably binding antibody variants (the sought-after antibodies) from the serum.

### Example 6: Binding specificities of the affinity-purified antibodies

Western blotting demonstrated that the affinity-purified antibodies had preserved their binding specificity for laminin P1 and that the different preparations reacted more strongly with unreduced laminin P1 than with the linear laminin P1 fragments which were separable by reducing the disulfides (Gerl, M. et al. (1991) Eur. J. Biochem. 202:167-174). This is circumstantial evidence for a conformation-dependent component in the binding specificity of the antibodies. It was furthermore found that the four antibody preparations differed in their binding preferences. The two antibody preparations R3 and R4, which were obtained by immunizing with conjugate 1, recognized identical laminin P1 bands which were obtained after subjecting the unreduced sample to SDS (sodium dodecyl sulfate) gel electrophoresis. While the two antibody preparations R2 and R905 (anti-conjugate 2) also exhibited reaction patterns which were identical to each other, they recognized fewer laminin P1 bands than did the two anti-conjugate 1 antibody preparations.

In the immunochemical detection of the laminin P1 bands which were separated by reduction and SDS gel electrophoresis, it is striking that all four antibody preparations exhibited different binding preferences for laminin γ1 Ill 4-containing fragments.

### Example 7: Inhibition assays—Inhibiting laminin/nidogen binding with affinity-purified antibodies

The inhibitory activity of the affinity-purified antibodies can be identified by means of a coated-tube assay which measures the binding of radioactively labeled nidogen to (human placental) laminin P1-coated tubes in the presence of the antibodies.

Radioactively labeling nidogen with ¹²⁵iodine

Recombinantly produced human nidogen (35 μg, for description of the clone and culturing and purification conditions, see Mayer, U. et al. (1995) Eur. J. Biochem. 227:681-686) was dissolved in 250 μl of PBS, and 0.405 mCi (=15 Mbq) of Na iodide (¹²⁵I) solution (=1.55 μl, Nordion Europe), 10 μl of 0.5 M Na phosphate, pH 7.4, and 40 μg of chloramine T (N-chloro-4-toluenesulfonamide sodium salt, Merck) in 100 μl of 0.05 M Na phosphate, pH 7.4, were added to the solution. The reaction took place for 60 seconds at room temperature and was stopped by adding a solution of 40 μg of Na metabisulfite (Riedel-de-Haen) in 100 μl of 0.05 M Na phosphate, pH 7.4. This addition was made in at most 30 seconds and 900 μl of 1% BSA (Sigma) in PBS were then added to the mixture. Free radioactivity and excess salts were separated off by means of molecular sieve chromatography using a PD 10 column (Pharmacia). The iodinated nidogen, which was eluted in PBS, was pooled and diluted with 1% BSA/0.05 M Na phosphate/0.01% Na azide, pH 7.4, such that a concentration of 50 ng/ml was obtained.

Coating reaction tubes

Reaction tubes (Greiner, 75×12, No. 115061) are coated at 4°C. overnight with a laminin P1 solution, 4 μg/ml in carbonate buffer (0.159 g of Na₂CO₃; 0.293 g of NaHCO₃; 0.02 g of NaN₃ in 1 liter of distilled water), 20 μg/ml BSA (bovine serum albumin, Serva), pH 9.2. Free binding sites are then blocked by incubating with 0.5 ml of 0.5% BSA in PBS/0.04% Tween 20 for 2 hours.

Inhibition assay using “laminin-mimetic” structures (sequential inhibition) 200 μl of iodinated nidogen (approx. 10 ng, approx. 40,000 counts per min) and 200 μl of the inhibitor (e.g. peptides which can be derived from the laminin γ1 III 4 domain) or standards (laminin γ1 III 3-5) were shaken in a reaction vessel at room temperature. Both the inhibitor and the standard were dissolved in PBS/0.04% Tween 20. After an incubation period of 3 hours, 150 μl were transferred from this mixture into the coated tubes and incubated at room temperature for a further 2 hours. Finally, the solution was tipped out and the tubes were washed twice with 1 ml of PBS/0.04% Tween 20, after which the bound radioactivity (nidogen) was measured in a gamma counter. The quantity of bound nidogen in the solutions containing inhibitor was related to that of the nidogen when no inhibitor was added.

Inhibition assay using “nidogen-mimetic” structures (sequential inhibition) 150 μl of the inhibitor (e.g. an antibody which binds to the laminin γ1 III 4 domain or a peptide which can be derived from the nidogen sequence) or standard (recombinant nidogen) were shaken for 3 hours in the laminin P1-coated reaction vessels. Both inhibitor and standard were dissolved in PBS/0.04% Tween 20. After the sample had been sucked off, 150 μl of iodinated nidogen (approx. 10 ng, approx. 40,000 counts per min) were added for a period of 2 hours in order to displace the bound inhibitor. Finally, the solution was tipped out and the vessels were washed twice with 1 ml of PBS/0.04% Tween 20, after which the bound radioactivity (nidogen) was measured in a gamma counter. The quantity of bound radioactive nidogen was related to the concentration of the inhibitor or concentration of the standard.

Inhibition assay (simultaneous inhibition)

In this assay variant, there was no preliminary incubation. Instead, 75 μl of iodinated nidogen (10 ng) together with 75 μl of inhibitor or standard were pipetted directly into the coated tubes, which were then incubated at room temperature for 2 hours; otherwise, the procedure was analogous to that used for the sequential assay variants.

Result

An IC_{50%} of 0.22 nM, with a standard deviation of +/−15%, was obtained for the laminin γ1 III 3-5 standard from ten independent assays. The IC_{50%} is defined as the concentration of the substance which is required in order to inhibit the binding of nidogen to laminin P1 by 50%. For comparison, an IC_{50%} of 0.05 nM, with a standard deviation of +/−52%, was obtained using the (equilibrium) inhibition assay described in U.S. Pat. No. 5,493,008.

Table 2 shows the IC₅₀% values of the antibody preparations R3, R905, R1.2, R2.2 and R3.2 and of different free peptides. Antibody preparations R1.2, R2.2 and R3.2 derive from a second round of rabbit immunizations with new conjugate II and comparable purification. The results provide evidence of the reproducibility of the method.

TABLE 2
Inhibition of laminin/nidogen binding by the antibodies according
to the invention.

| | | | | | | Peptide | Peptide |
|---|---|---|---|---|---|---|---|
| Inhibitor | R3 | R905 | R1.2 | R2.2 | R3.2 | (1)* | (2)* |
| | IC_{50%} | IC_{50%} | IC_{50%} | IC_{50%} | IC_{50%} | IC_{50%} | IC_{50%} |
| | nM | nM | nM | nM | nM | nM | nM |
| sequential** | 72 | 150 | 500 | 80 | 350 | 60000 | 20000 |
| simultaneous* | 110 | — | 600 | 80 | 500 | 60000 | 20000 |

*Amino acid sequence, see Table 1
**Assay type

U.S. Pat. No. 5,493,008 gives IC_{50%} values of between 22 nM and 1000 nM for inhibitory peptides which can be derived from the nidogen-binding domain. Because of the drastically shortened incubation times, it was not possible to achieve these values with the assay which was selected; for example, in the assay described here, the peptide DNIDPNAVGNL only achieved an IC_{50%} of 60000 nM.

### Example 8: Characterization of the binding kinetics using the BIAcore® system

Biospecific interactions can be monitored on-line using the BIAcore® system from Pharmacia Biosensor. The principle of the measurement is based on an optical phenomenon (surface plasmon resonance) which is affected by the mass which is bound on a gold film. Expressed in simple terms, the system is miniaturized affinity chromatography on a gold sensor surface. The quantity of specifically bound ligand can be depicted visually in the form of a resonance signal (Chaiken, I. et al. (1992) Anal. Biochem. 201:197-201; Karisson, R. et al. (1992) in: Structure of Antigens; (Ed.: van Regenmortel). pp. 127-148; CRC Press, Boca Raton, Fla.)

Laminin P1 was immobilized, at a concentration of 200 μg/ml in 10 mM Na acetate, pH 4.0, on the sensor chip in accordance with the instructions in the user manual. A matrix containing 4000 RU of bound laminin P1 is obtained. A double impulse of in each case 4 μl of 100 mM HCl can be carried out in order to regenerate the affinity matrix.

At a flow rate of 2 μl/min in HBS buffer (10 mM HEPES, 3.4 mM EDTA, 150 mM NaCl, 0.005% BIAsurfactant P20; pH=7.4), nidogen (20 μg/ml) bound to laminin P1 with parabolic saturation kinetics, the affinity-purified antibody preparations R905 and R3 in linear dependence on the antigen. The fact that it was still not possible to observe any transition to the equilibrium state after 1400 seconds can be an expression of the fact that the specific peptide sequence of laminin P1 which is responsible for binding nidogen was readily available to the antibodies. The antibodies bound to this sequence irrespective of whether it was in the biologically active conformation or in a different conformation. Evidence for this is provided by the observation that the nidogen displayed a clear transition to the saturation phase after only binding 600 RU. Evidently, therefore, not all the immobilized laminin P1 molecules were present in a structure which was recognizable to the nidogen since the theoretical maximum saturation of the layer, of 2500 RU, was not reached. This saturation would, however, have been reached by the two antibodies after a long contact time. It is striking that the R905 sample had to be used at a tenfold higher concentration (320 μg/ml) in order to achieve a binding rate which was comparable to that for R3 (33 μg/ml). On the other hand, the binding of R905 to laminin P1 was more stable than that of R3 since the rate at which R905 dissociated (recognizable from the time of the change to the HBS buffer: 1500 seconds) was substantially slower than that at which preparation R3 dissociated.

These findings explain the differences between R3 and R905 which were observed in the inhibition assays:

Antibody preparation R3 inhibits laminin/nidogen binding better than does R905 because the R3 binding is characterized by more rapid association kinetics.

Antibody preparation R3 also inhibits when it is incubated simultaneously with nidogen in the laminin P1-coated tubes because it has good association kinetics at a concentration which is comparable to that of the nidogen.

Antibody preparation R905 is only able to inhibit in the “sequential inhibition” assay variant because it is characterized by a slow dissociation rate and can therefore no longer be displaced so readily from the antigen by the nidogen which is subsequently added. When R905 and nidogen are competing simultaneously for the binding site, R905 is inferior to nidogen due to its very slow association kinetics.

### Example 9: Detecting the binding specificities of the affinity-purified antibody preparations R3 and R905 by means of Western blotting

The interaction of antibody preparations R3 and R905 with conjugate 2 and ovalbumin were investigated in Western blot analyses. For this, 4-12% NuPAGE™ gels (NOVEX™, San Diego, Calif.) and a MOPS buffer were used, in accordance with the NOVEX™ instructions, to fractionate the antigens, which were then transferred to nitrocellulose membranes using NUPAGE™ transfer buffer (NOVEX™). The antigens were incubated with the test antibodies after free binding sites on the membrane had been blocked with 1 μg/ml polyvinyl alcohol (1 min). Bound antibodies were then detected with anti-rabbit IgG antibodies to which the enzyme alkaline phosphatase was covalently bonded.

It was found that the affinity-purified antibodies bind exclusively to the peptide since it is not possible to detect any interaction with the carrier protein ovalbumin. It was likewise not possible to observe any reaction with the blue “See Blue” standard markers (NOVEX™).

It was also demonstrated that both R3 and R905 react with laminin and laminin derivatives from different species (human placental laminin and rat yolk sack laminin (Calbiochem), human placental laminin P1 and mouse EHS tumor laminin P1, and also recombinantly prepared mouse laminin γ1 III 3-5). This provides evidence in support of the antibodies binding to the conserved sequence within the nidogen-binding domain. Neither antibody preparation exhibited any crossreactivity with human nidogen or human collagen type IV. This is the prerequisite for the unambiguous use of the above-described antibodies as nidogen antagonists.

### Example 10: Preparation of monoclonal antibodies

In order to obtain monoclonal antibodies, suitable vertebrates, preferably mice or rats, are immunized, for example, with laminin, laminin P1, laminin γ1 III 3-5 or laminin γ1 III 4, or with the conjugates cited in Example 2, using standard methods. When the antiserum exhibits a specific immune reaction, standard methods are used to isolate MAb-producing hybridomas.

Binding assays (e.g. dot blotting or Western blotting using laminin γ1 III 3-5 and/or laminin γ1 III 4) and, in particular, the inhibition assays described in Example 7 as well, and other methods, are used to screen for the desired antibodies or the corresponding hybridoma clones. The selected clones constitute a source for synthesizing large quantities of the antibodies according to the invention.

Customary methods, for example binding to protein G or protein A, can be used to purify the desired antibodies. However, affinity chromatography on laminin P1 columns is preferably carried out. As in the case of the above-described polyclonal antibodies, this purification step makes it possible to choose and selectively concentrate the monoclonal antibodies which have the best binding constants.

An example of an antibody according to the present invention is the monoclonal antibody (MAb) which is produced by the monoclonal cell clone A6/2/4 which was deposited under deposition number DSMACC2327 in the Deutsche Sammiung von Mikroorganismen und Zelikulturen GmbH (German Collection of Microorganisms and Cell Cultures) (DSMZ, Marscheroder Weg 1b, 38124 Braunschweig, Germany) on Oct. 27, 1997 in accordance with the provisions of the Budapest treaty.

The hybridoma derives from immunizing mice with laminin P1 which had been isolated from human placenta. The purification of the antigen, and the production of the hybridomas, is described in EP 0 696 597 A2. Antibody A6/2/4 was identified on the basis of the characteristics of its binding to laminin γ1 III 3-5 and laminin γ1 III 4. It is a monoclonal antibody of the IgM subtype which can be purified by means of molecular sieve chromatography. Partly because it is polyvalent, it binds extremely strongly to laminin P1. For this reason, and due to the size of the IgM antibody, it is not possible to elute it from laminin P1 affinity columns (see above). The strong binding to laminin P1 is reflected in the (“simultaneous variant”, see above) inhibition assay. MAb A6/2/4 is able to inhibit the laminin/nidogen association with an IC₅₀ of 30 nM.

Due to the pronounced conformation-dependence in its binding, the binding epitope in the laminin γ1 III 4 domain (the nidogen-binding domain of laminin) cannot be circumscribed unambiguously. However, the fact that peptides which can be derived from the nidogen-binding sequence of laminin partially (75-80%) suppress the interaction of the antibody with laminin P1 indicates that the binding epitope of MAb A6/2/4 overlaps with that of nidogen.

The preparation of inhibitory, monoclonal antibodies which, like the polyclonal antibodies according to the present invention, can be generated using the peptide/ovalbumin conjugate is described below.

Mice of the SJL/J strain are immunized subcutaneously with 50 μg of peptide 2/ovalbumin conjugate (conjugate 2, see above) in the presence of complete Freund's adjuvant. After 4 and 8 weeks, the immune reaction is boosted by further subcutaneous injections each of 25 μg of conjugate 2 in the presence of incomplete Freund's adjuvant, and a further 7 weeks is allowed to elapse. Three days before the fusion, the immune response is boosted by intraperitoneal injection of a further 25 μg of conjugate 2.

For the fusion, the animals are sacrificed and the spleen cells are isolated. The spleen cells are fused with the myeloma cell line P3X63AG8.653 in the presence of polyethylene glycol. Selection for spleen cell×P3X63AG8.653 hybrids takes place by cultivating the fusion mixture in hypoxanthin/aminopterin/thymidine medium for a period of three weeks. To obtain a stable cell line, the resulting cell clones are subcloned several times. The resulting cell colonies are tested for antibody production in various immunological binding assays. The resulting cell lines E79/1/6 and E82/1/10 were selected on the basis of the screening strategy below.

Experiments to characterize and identify the specific monoclonal antibodies

Immunization of SJL/J mice with conjugate 2 leads to an extremely large number of antibody-producing hybridoma clones. The antibodies in the culture supernatant show a strong immune reaction with laminin γ1 III 3-5, laminin P1 and ovalbumin.

In order now to find clones which produce monoclonal antibodies against the laminin nidogen-binding domain with the native structure it was necessary to carry out a suitable screening method.

In this screening method, attention is principally directed at the binding of the antibodies to laminin P1 and laminin γ1 III 3-5. It is necessary at the same time during the subclonings to take care that the reaction with the carrier protein ovalbumin is negligible and antibodies which recognize ovalbumin exclusively are selected.

Tables 3 and 4 show the results obtained with two clones (E79 and E82) identified in this way.

TABLE 3
Determination of the binding of E79 in an ELISA

| | Laminin | | |
|---|---|---|---|
| | _{γ}1 III 3-5, | Laminin P1 | Ovalbumin |
| | Coating: | Coating: | Coating: |
| | 2.5 μg/ml | 2.0 μg/ml | 20 μg/ml |
| Hybridoma E79 | 1.33 | 0.68 | 1.63 |
| undiluted culture | | | |
| supernatant | | | |
| E79/1/6, | 2.03 | 0.16 | 0.27 |
| 1st cloning | | | |
| undiluted culture | | | |
| supernatant | | | |
| E79/1/6, | 0.56 | 0.33 | 0.05 |
| purified antibody | | | |
| 2.5 μg/ml | | | |

TABLE 4
Determination of the binding of E82 in an ELISA

| | Laminin | | |
|---|---|---|---|
| | _{γ}1 III 3-5, | Laminin P1 | Ovalbumin |
| | Coating: | Coating: | Coating: |
| | 2.5 μg/ml | 2.0 μg/ml | 20 μg/ml |
| Hybridoma E82 | 1.77 | 1.48 | 2.33 |
| undiluted culture | | | |
| supernatant | | | |
| E82/1/10, | 1.32 | 0.26 | 0.05 |
| 1st cloning | | | |
| undiluted culture | | | |
| supernatant | | | |
| E82/1/10, | 1.55 | 0.5 | 0.18 |
| purified antibody | | | |
| 6.4 μg/ml | | | |

It has evidently been possible by the cloning to separate out cells which show an immune reaction with ovalbumin. It was at the same time possible in each case to separate a cell clone which produces antibody which shows binding to laminin γ1 III 3-5 and laminin P1. This fact, and the fact that the immunization took place with a peptide derived from the laminin nidogen-binding domain, demonstrates that the antibodies found bind to the laminin nidogen-binding motif with the native structure.

Direct proof of the binding specificity is provided by the “simultaneous” variant of the inhibition assay (see above), in which the antibodies found compete directly with iodinated nidogen for binding to the nidogen-binding motif for intact laminin (laminin from mouse EHS Tumor, Chemicon, No. CC095).

The antibody (IgG2a subtype) produced by the cell clone E79/1/6 inhibits the laminin-nidogen association with an IC50 of 19 nM, and the antibody (IgG1 subtype) produced by the cell clone E82/1/10 inhibits the laminin-nidogen association with an IC50 of 190 nM.

1. An antibody, or part thereof, which binds to the nidogen-binding laminin γ1 III-4 domain.

2. An antibody, or part thereof, as claimed in claim 1, which binds to the highly conserved region of the a loop or of the a and c loops of the nidogen-binding laminin γ1 III-4 domain, or in the immediate vicinity of these loops.

3. An antibody, or part thereof, as claimed in claim 2, which binds, in a conformation-dependent manner in relation to the epitope, directly, or in an overlapping manner, to the highly conserved region of the a loop or of the a and c loops.

4. An antibody, or part thereof, as claimed in one or more of claims 1 to 3, which binds at least to a peptide as depicted in Table 1.

5. An antibody as claimed in one or more of claims 1 to 4, which is polyclonal.

6. An antibody as claimed in one or more of claims 1 to 4, which is monoclonal.

7. An antibody as claimed in claim 6, which is a chimeric, humanized, bispecific or oligospecific antibody.

8. An antibody as claimed in one or more of claims 1 to 7, which inhibits laminin/nidogen binding competitively or partially competitively.

9. An antibody which can be obtained by immunizing immunocompetent vertebrates with laminin or laminin P1, as immunizing antigen, and subsequently identifying the antibody using laminin γ1 III-3-5 and/or laminin 1 III-4 and testing this latter antibody for its ability to inhibit laminin/nidogen binding competitively or partially competitively.

10. An antibody which can be obtained by immunizing immunocompetent vertebrates with laminin γ1 III-3-5, as immunizing antigen, subsequently identifying the antibody using laminin and/or laminin P1 and testing the latter antibody for its ability to inhibit laminin/nidogen binding competitively or partially competitively.

11. An antibody which can be obtained by immunizing immunocompetent vertebrates with laminin γ1 III-4 and/or with peptides which contain important constituents of the nidogen-binding sites but do not contain the complete amino acid sequence of the laminin γ1 III-4 domain, as immunizing antigen.

12. An antibody as claimed in claim 11, wherein laminin γ1 III-4 is used as immunizing antigen.

13. An antibody as claimed in claim 11, wherein one or both the peptides depicted in Table 1 is/are used as immunizing antigen.

14. An antibody as claimed in one or more of claims 11 to 13, which is identified using laminin and/or laminin P1.

15. An antibody as claimed in one or more of claims 11 to 14, which is tested for its ability to inhibit laminin/nidogen binding competitively or partially competitively.

16. An antibody as claimed in one of claims 9 to 15, wherein monoclonal antibody-producing hybridoma cells are generated.

17. An antibody as claimed in one of claims 9 to 16, which is purified from antibody-containing material by means of affinity chromatography.

18. An antibody as claimed in claim 18, wherein the affinity chromatography is carried out on laminin and/or laminin P1 as affinity matrix.

19. A cell or cell line, which produces an antibody, or parts thereof, as claimed in one or more of claims 1 to 8.

20. The hybridoma DSMACC2327.

21. An antibody which is produced by the hybridoma DSMACC2327.

22. A process for preparing an antibody as claimed in one of claims 1 to 8, which comprises immunizing immunocompetent vertebrates with laminin or laminin P1, identifying the antibody using laminin γ1 III-3-5 and/or laminin γ1 III-4 and testing this antibody for its ability to inhibit laminin/nidogen binding competitively or partially competitively.

23. A process for preparing an antibody as claimed in one of claims 1 to 8, which comprises immunizing immunocompetent vertebrates with laminin γ1 III-3-5, identifying the antibody using laminin and/or laminin P1 and testing this antibody for its ability to inhibit laminin/nidogen binding competitively or partially competitively.

24. A process for preparing an antibody as claimed in one of claims 1 to 8, which comprises immunizing immunocompetent vertebrates with laminin γ1 III-4 and/or with peptides which contain important components of the nidogen-binding sites but do not contain the complete amino acid sequence of the laminin γ1 III-4 domain.

25. The process as claimed in claim 24, wherein laminin γ1 III-4 is employed as immunizing antigen.

26. The process as claimed in claim 24, wherein one or both of the peptides depicted in Table 1 is/are employed as immunizing antigen.

27. The process as claimed in claim 26, wherein the immunizing antigen is coupled to a carrier.

28. The process as claimed in one or more of claims 24 to 27, wherein the antibody is identified using laminin and/or laminin P1.

29. The process as claimed in one or more of claims 24 to 28, wherein the antibody is tested for its ability to inhibit laminin/nidogen binding competitively or partially competitively.

30. The process as claimed in one or more of claims 22 to 29, wherein monoclonal antibody-producing hybridoma cells are generated.

31. The process as claimed in one or more of claims 22 to 30, wherein the antibody is purified from antibody-containing material by means of affinity chromatography.

32. The process as claimed in claim 31, wherein the affinity chromatography is carried out on laminin and/or laminin P1 as affinity matrix.

33. An antibody, or part thereof, as claimed in one of claims 1 to 18 or 21 for use as a pharmaceutical.

34. A pharmaceutical which comprises one or more antibodies or antibody parts as claimed in at least one of claims 1 to 18 and 21.

35. The use of one or more antibodies or antibody parts as claimed in at least one of claims 1 to 18 and 21 for preparing a pharmaceutical for treating diseases which are characterized by an increased or undesirable synthesis of basement membranes.

36. The use as claimed in claim 35, wherein the disease is a form of diabetic late complications which are accompanied by thickenings of the basement membrane, a form of arteriosclerosis, a fibrosis or a disease in which angiogenesis contributes to aggravation of the clinical picture.

37. The use as claimed in claim 35 or 36 for preparing a pharmaceutical for treating diabetic retinopathy, alcoholic hepatic fibrosis, pulmonary fibrosis, a cancer disease, diabetic nephropathy or a disease having a strong inflammatory component such as rheumatoid arthritis, osteoarthritis, vasculitis, hemangiomas and psoriasis.

38. An antibody, or part thereof, as claimed in one of claims 1 to 18 or 21 for use as a diagnostic agent.

39. A diagnostic agent which comprises one or more antibodies or antibody parts as claimed in at least one of claims 1 to 18 and 21.

40. The use of one or more antibodies or antibody parts as claimed in at least one of claims 1 to 18 and 21 for preparing a diagnostic agent for detecting γ1-containing laminin isoforms in biological samples, body fluids or tissues.

41. The use of an antibody, or a part thereof, as claimed in one of claims 1 to 18 or 21 as an aid in biological and pharmacological models for developing and evaluating substances which affect the laminin/nidogen interaction.

42. The use as claimed in claim 41 in a biological and pharmacological model for developing and evaluating substances which affect the laminin/nidogen interaction.
